(19) United States
(12) Patent Application Publication
(10) Pub. No.: US 2011/0139669 A1
(43) Pub. Date: Jun. 16, 2011

(54) Preparing of products, in particular cellulose-containing products and/or spaces

(75) Inventors: Hans-Juergen Huppert, Leipzig (DE); Colin Berrido, Bagshot (GB)

(73) Assignee: Bell Flavors & Fragrances Duft und Aroma GmbH, Leipzig/Miltitz (DE)

(21) Appl. No.: 12/308,780

(22) PCT Filed: Jun. 25, 2007
(86) PCT No.: PCT/EP2007/005618

§ 371 Date: Dec. 23, 2008

(30) Foreign Application Priority Data
Jun. 24, 2006 (DE) 10 2006 029 092.5

(51) Int. Cl. B65D 85/00; A61K 8/31; A61K 47/06; A23L 1/22; A23P 1/08; A23L 1/025

(52) U.S. Cl. 206/524.1; 512/1; 514/772; 426/650; 426/307; 426/241; 426/238; 426/234

(57) ABSTRACT

The process of the invention for the preparation of products, in particular cellulose-containing products (1) and spaces includes the arrangement of at least one flavour and/or fragrance (3) and/or active substance, bound in a solid carrier material, in and/or at the product (1) and/or the direct proximity of the cellulose-containing product (1) and/or the space. Furthermore, means are provided for the preparation of products, in particular cellulose-containing products, and of spaces as described, as well as systems from these means and products, in particular cellulose-containing products.

The invention is a process for the preparation, i.e. flavouring and/or the transfer of active substances to products, in particular cellulose-containing products such as cigarettes as well as handkerchiefs, napkins, kitchen towels and other cellulose-based products, and to spaces. Furthermore the invention concerns means for the flavouring and/or transfer of active substances to products, in particular cellulose-containing products, and spaces as well as a system from the means and products, in particular cellulose-containing products.

State of the art publications describe a number of options for the flavouring of cellulose-containing products. Thus the German Utility Patent DE 202 004 007 682 U1 refers to flavoured towels and bags where flavouring is achieved by spraying, misting, gassing or soaking the products themselves or their source materials in flavour-releasing fluids or solutions.

In a similar way, a process for the production of flavoured stranded tobacco material described in the German Patent DE 38 21 677 C2 involves a liquid medium comprising a flavoured substance that is sprayed on the tobacco. The disadvantage of the aforementioned methods for the flavouring of cellulose-containing products is that additional and relatively complicated process steps are required during the manufacture of these products.

Furthermore, German Disclosure (Offenlegungsschrift) DE 44 03 018 A1 describes a filter for cigarettes, pipes or similar devices where the tobacco smoke is flavoured by an aromatic substance absorbed in the wrapping for the filter material and flavouring the smoke when passing the filter. The disadvantage of this indirect way of flavouring is that the aromatic substance inserted in the filter wrapping evaporates relatively quickly during the storage of the filters or the cigarettes equipped therewith, so that flavouring is perceived as adequate only as long as the cigarettes are fresh, but vanishes almost completely and is hardly noticeable after a short period of time.

Another established method is to put chemically or biologically acting additives (active substances) like insecticides and similar materials onto cellulose-containing products by spraying the additives in order to impart to the product, by means of the active substance, certain characteristics. In this case as well, there is a problem created by the necessity to add the active substances during an additional process step and that a diminished effect has to be expected in the course of time.

Therefore the underlying task of the present invention is to develop a process for the flavouring and/or the transfer of active substances to products, in particular cellulose-containing products ensuring even and long-lasting flavouring and the continuous effect of the active substances in products without requiring any additional and complicated process steps during their manufacture. Furthermore, this flavouring and transfer of active substances shall also be usable for the preparation of spaces.

In accordance with the invention, this task is solved by the characteristics of claim 1. Suitable variations of this subject of the invention form the characteristics of subclaims 2 through 16.

The invention is based on the knowledge that products, in particular cellulose-containing products, can be combined with those volatile substances in a much more efficient and long-lasting manner if the said volatile substances are bound in a solid carrier material and can be combined with the product irrespective of its manufacturing process.

This process of flavouring and/or the transfer of active substances to products, in particular cellulose-containing products, is not only highly efficient as the volatile substances are continually released over a long period of time, it is also very cost-effective as the substances do not need to be added to the product. The volatile substances can be arranged in proximity to the product but they should be located sufficiently close to the product so that an adequate amount of the substances can be transported to the product. It goes without saying that they can be positioned in direct contact with or inside the product (e.g. in a folded towel). The volatile substances can also be positioned together with the product inside one product packaging. For the flavouring of spaces or the transfer of active substances to spaces the volatile substances are arranged in the space. In this manner the flavouring process and/or the transfer of active substances is highly flexible and can be adapted to e.g. seasonal requirements and market-related changes in requirements.

Examples of solid carrier materials are defined in DE 10 2004 052 929 A1 as well as DE 102 47 583 A1 the contents of which with regard to carrier materials and their use is included here in all points. The important thing is that these are carrier materials that bind the flavour, fragrance or active substances. This means it is a liquid flavour, fragrance or active substance liquid at normal temperature bound in the solid carrier material by adsorbing or absorbing the flavour, fragrance or active substance to the carrier material. The fragrance is encapsulated by the carrier material and then slowly released. This is what distinguishes it, for instance, from so called fragrance trees for automobiles and similar things where a felt (porous and absorbent carrier) has been soaked with a fragrance.

For instance, the fixed carrier material with at least one flavour, fragrance or active substance bound therein can be used in particulate form such as powder, granulate, chips or pearls. On the other hand, the fixed carrier material with at least one flavour, fragrance or active substance bound therein can be applied to or introduced in a support material, where preferably a flat material, above all paper, cardboard, plastic sheeting or metal foil, ceramics, glass, non-woven fabric or tissue or a tissue-like product from textile material, glass, ceramics, metal or plastics fibres are used. The support material may also be a part of any shape created by basic forming or transforming or combined from several elements. The coating of the support material with the solid carrier material with at least one flavour, fragrance or active substance bound therein can be performed in such a way that a melt of the carrier material with at least one flavour, fragrance or active substance bound therein is applied to or inserted in the support material and subsequently solidified by cooling. Application can be done by coating, spraying, soaking or pouring.

Preferably, the inner side of the packaging material itself can be used as support material for the application or insertion of the carrier material with at least one flavour, fragrance or active substance bound therein.

It is especially preferred that the carrier material is sealed off to the environment or the product by an enclosure impermeable to the carrier material or the carrier material and support material, which however is permeable for the flavouring or active substances. (It is however sufficient if the enclosure is permeable for the fragrance, flavour or active substance only in certain desired areas. For instance, a one-sided application of a permeable area of the enclosure is sufficient on a packaging which is itself as support material if the packaging is impermeable for the carrier material.) Thus the flavouring or active substance can pass through the enclosure in order to flavour the product or space or to provide them with active substances, however neither the carrier material nor the support material can come into contact with the product or the environment so that specific safety, in particular for children, is ensured. This enclosure can comprise or fully consist of paper, polyethylene, in particular modified polyethylene such as ethylene ethyl acetate (EEA) or ethylene vinyl acetate (EVA), normal PE such as low density polyethylene or high density polyethylene or silicone sheet.

Furthermore it may be envisaged that the packaging comprises a material impermeable to the flavour, fragrance or active substance, such as cellophane, which is in particular connected with the packaging as one piece. This means that the flavouring of the product or the provision of the product with an active substance stays active and fresh for a longer period of time. Naturally, this material may itself form the packaging.

The process of the invention is basically also very well suitable for the preparation of spaces, such as rooms, but also small enclosed spaces such as washing machines, dish washers, laundry driers and refrigerators etc. In the case of driers and washing machines it is clear that above all the clothes contained therein are to be prepared rather than the inside of the appliances, however the latter may also be an intended purpose.

Therefore, volatile substances in the framework of the present invention are flavouring substances such as flavours and fragrances as well as chemically and/or biologically acting substances.

The volatility of the flavouring substances creates a sensual perception via corresponding receptors (e.g. in the nose). To give an example, perfumes consist of top, heart (or middle) and base notes. The said three notes include different fragrance components, which differ in their volatility with an increase of volatility from top, heart (or middle) and base notes. These three notes include different fragrance components which differ with respect to their volatility which increases from the base note via the heart note to the top note. On account of these different volatility levels the odour sensation varies after prolonged storage. In the same way volatility of the flavouring ingredients leads to a decrease in their intensity.

The process of the invention enables these flavouring ingredients to be freshened and to be imparted to the product and/or the space again and again. This is achieved by the capacity of the solid matter not only to store and preserve the flavouring matter but also to deliver it gradually in a controlled manner.

Active ingredients under the present invention are substances imparting a chemical and/or biological effect to a product. Examples of those active substances include insecticides, antibacterial substances, antifungal or antiputrefactive substances, preservatives, therapeutical, i.e. pharmaceutical substances and also substances influencing or controlling animals like pheromones, hormones or the essential citronella oil used as a gnat repellent.

The volatility of these chemical or biological substances means that their biological and/or chemical effectiveness diminishes after some time say by lowering of the concentration or due to environmental changes of the substances like by oxidation and similar processes.

Due to the process under this invention the aforementioned substances can be provided in such a manner that they are supplied fresh to the product and/or the space on an ongoing basis and thus develop a long-lasting effect. Thus the products or spaces have a prolonged chemical and/or biological activity.

Another especially advantageous further improvement of the method described in this invention is the acceleration of the process of flavouring and/or imparting an active substance by the introduction of energy. Principal energy sources include electromagnetic radiation in the infrared, visible and ultraviolet spectrums, but also microwave radiation and ultrasound. Preferred sources are, however, microwave and heat radiation by which the accelerated release of flavour or active ingredients from the support material is facilitated.

In this manner it is possible to prepare very quickly such products which are distributed after opening their packaging and which, at that time, should present their optimal flavouring condition and their highest ingredient activity. An example are towels distributed after meals by the flight attendants of airlines. These towels can be prepared by microwave treatment on the ground or during the flight so that the airline passenger after receiving them perceives a particular feeling of freshness.

Independent protection is also claimed for means for the flavouring of products, in particular cellulose-containing products and/or spaces and/or for the transfer of active substances to products, in particular cellulose-containing products or spaces in accordance with claim 17.

The means of the invention consist of a solid carrier material in which at least one fragrance, flavour or active substance is bound, and an enclosure where the carrier material is accommodated, with the enclosure being permeable for the fragrance, flavour or active substance in at least one location. The enclosure fully enclosing the carrier material is impermeable for the latter.

The carrier material with the fragrance, flavour or active substance bound therein can be used in different forms and shapes: on the one hand in a particulate form such as granulate, balls, chips etc, with an outer diameter of up to 10 mm, preferably up to 5 mm, or as a powder in a range of 10 μm to 100 μm, preferably 25 μm to 50 μm, or on the other hand in a film shape. A film shape means in that context that the carrier material with the fragrance, flavour or active substance bound therein is applied to or inserted in a support material. Paper, cardboard, plastics, ceramics or glass, non-woven fabric or tissue or tissue-like products from textile material, glass, ceramics, metal or plastics fibres, or laminates of these substances, can be used as support material. These support materials may be used as flat material or as a part of any shape created by basic forming or transforming or combined from different elements. Typically such film shapes (films) are up to 3 mm, preferably 2 mm thick. In this context we would like to refer to DE 10 2005 036 415 A1, the content of which is fully integrated in this specification with regard to the film shape and its manufacture.

Certain characteristics are linked with these forms and shapes.

In the case of granulates, the fragrance, flavour or active substances are to a high degree encapsulated in the carrier material. Therefore energy is required in order to quickly release these substances. These substances are best released from granulates at temperatures of 50° C. to 55° C., however there is the danger that the carrier material melts and contaminates or even damages the surroundings. Otherwise, loose pastilles can be easily scattered and carried away from the preferred application environment.

In the case of the film shape, the encapsulation is not so strong as in the case of granulates since the carrier material is arranged with the bound fragrance, flavour and active substance as a thin film layer on the support material, and thus the fragrance, flavour and active substances reach the surface much faster. This means that fast release can be reached already at room temperature, however, damage may occur during direct contact of the environment with the film shape (e.g. when touching it with the hand or during contact with other objects).

The powder is a very fine powder which is generated, for example, by cryogenic milling of a carrier material present in solid form, with the bound fragrance, flavour or active substance under a liquid nitrogen atmosphere (or carbon dioxide gas or dry ice). Other manufacturing methods are also applicable. Due to its enlarged surface, the powder releases the absorbed substances faster at room temperature than granulates or the film shape. Due to the small particle size, handling becomes however more difficult, and contamination of the surroundings is possible.

Of course, these forms and shapes may also be combined with each other. For instance, several forms and shapes may be included in a packaging in order to trans-fer certain fragrance, flavour and/or active substances. However, different forms and shapes may be introduced that contain the same fragrance, flavour and/or active substances.

To overcome these problems, the above-mentioned or other forms and shapes are, in accordance with the invention, enclosed in an enclosure that prevents the passage of the carrier material (and the support material that was possibly used) and is permeable for the fragrance, flavour or active substance at least in one area. This prevents emergence or contact of the carrier material (and the support material that may be used) in the environment as well as direct contact.

What is important is that the enclosure has to be permeable only in such areas where permeability is required for the flavour, fragrance or active substance. The other areas can be impermeable to fragrance, flavour or active substances, however emergence or contact of the carrier material (and the support material that may be used) in the environment or direct contact have to be prevented.

If permeability is required in every solid angle area, the enclosure must be permeable to the fragrance, flavour or active substance everywhere. If permeability is required only for certain solid angle areas, then the enclosure must be provided in a permeable way; a completely impermeable enclosure is sufficient in the other areas, however, the enclosure can be permeable everywhere which is after all a cost and design question.

If, for instance, the carrier material with a fragrance is applied to a packaging as a support material, then it is sufficient to provide the part of the enclosure that is permeable for the fragrance directly on the carrier material, as permeability is only required in these areas in order to flavour products located in the packaging. Then, as part of the enclosure, the packaging seals the carrier material towards the surroundings of the packaging, and the permeable part of the enclosure seals the carrier material towards the inner space of the packaging and thus towards the product(s) contained in the packaging.

With this enclosure, these means are very safe in their use which is above all very important from the point of view of safety for children.

A simple enclosure option meeting the requirements with regard to the permeability to these substances and simultaneous sealing of the carrier material is an enclosure made of paper, e.g. paper for tea bags, which is shaped to form a little bag. This is sufficient for granulates and the film shape since the paper retains these shapes well because of their size. However, it is less suitable for powders because of the small particle size of the powder grains. Furthermore there is the danger that the tea bag paper becomes torn due to its low mechanical strength. If, however, this is of no significance, tea bag paper may well be used.

As an alternative, polyethylene (PE), modified PE such as ethylene ethyl acetate (EEA) or ethylene vinyl acetate (EVA) as well as silicone may be used.

PE is a widely used favourable material which is non-polar and does not show good permeability for polar substances such as the aldehydes, ketones and esters normally found in flavours and fragrances. Nevertheless, there will be low permeability due to the very small interstices between the polyolefin particles in PE sheeting.

PE is available in different versions, for instance as LDPE (low density PE) and HDPE (high density PE). LDPE has a less packed molecular structure than HDPE which enables greater passage already at room temperature. In the case of HDPE, it is normally necessary to heat the substance to approx. 40° C. in order to ensure equally high permeability.

Alternatively, PE sheeting can be made permeable for polar substances by ensuring that a non-polar additive (solvent) is used which “guides” the substances through the sheeting. The underlying principle is that equivalent substances can dissolve equivalent substances. Since PE is a non-polar substance, non-polar hydrocarbons, for instance, are absorbed in the PE material which leads to swelling and enlargement of the polyolefin particles which means that the interstices between the polyolefin particles also become bigger, and a greater amount of the substance can penetrate through the PE sheeting.

An isoparaffinic solvent with the brand name Isopar from Exxon has turned out advantageous for use as a non-polar solvent. In particular Isopar L with a flash point of 62° C. is very well suited for this purpose. With regard to the total amount of solvent and flavours, fragrances and active substances, additions of this solvent to the fragrance, flavour or active substance of 0.5% to 50% are advantageous, while 10% is the preferred amount. However, it has to be taken into account that an excessive portion of solvents will lead to increased absorption in the polyolefin particles and thus increased swelling of the PE sheeting which may result in the formation of cracks. On the other hand, the use of solvents with a low flash point also leads to increased absorption and thus swelling so that again the danger of crack formation exists.

Adding polar solvents, in particular if these are of greater polarity than the fragrances, flavours or active substances, can also decrease permeability of the PE sheeting.

Another important influencing factor for the permeability of PE sheeting is sheet thickness, i.e. the thicker the sheeting, the lower the permeability. It is advantageous to use layer thicknesses of 0.1 mm to 2 mm, while 0.2 mm to 0.5 mm should be preferred.

Thus it has been shown that the amount of the substance that can permeate per time unit can be both enlarged and decreased which makes it possible to adapt the system to certain requirements.

Modified PE is normally more polar than pure PE, so that typical flavours and fragrances can pass without requiring specific solvents. Rather the substance itself acts like a polar solvent. In the case of this modified PE, polarity increases with increasing ethyl acetate or vinyl acetate content which means the permeability for polar substances is increased, however the strength of the modified PE decreases.

However, modified PE is more expensive and therefore less widely used. In the same way as normal PE sheeting, modified PE sheeting of this invention is preferably used in a temperature range from 5° C. to 80° C., in particular at 10° C. to 40° C. This means it is especially suitable for air fresheners.

Silicone sheet is advantageous for high-temperature applications up to 200° C., preferably of 80° C. to 120° C. For instance, sheet material with the brand name AltecSil from the British company Altec has proved valuable for fragrances and flavours.

Preferably, silicone sheet with a thickness from 0.1 mm to 1 mm is used, with permeability increasing with lower thicknesses. Typical applications are laundry driers and electrical air fresheners with higher temperatures.

Independent protection is also claimed for a system consisting of such means and one or several products, in particular cellulose-containing products in accordance with claim 25. Preferably, this system contains a packaging, in which both the product and the means are contained, where the packaging should preferably be impermeable to fragrances, flavours and active substances, in order to ensure long-lasting flavouring or provision of active substances.

The invention will be described below based on embodiments with reference to the Figures where

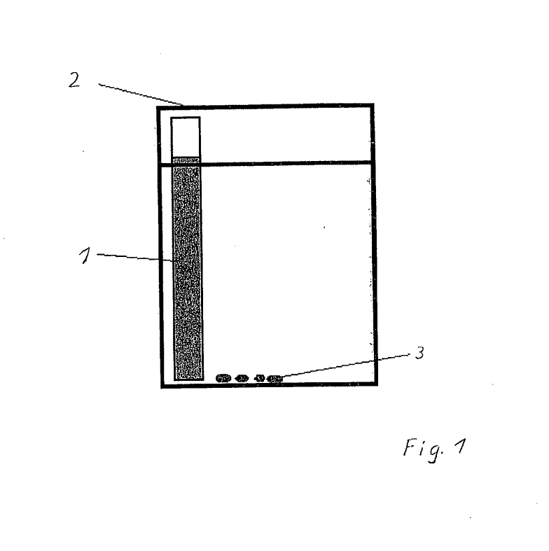

FIG. 1 shows cigarettes in a pack flavoured with ingredients bound in a bed of granular solid matter;

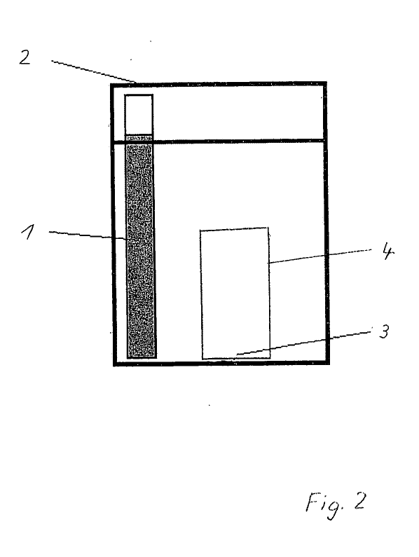

FIG. 2 shows cigarettes in a pack containing flavouring ingredients bound in a carrier material which is arranged in and/or onto a support material;

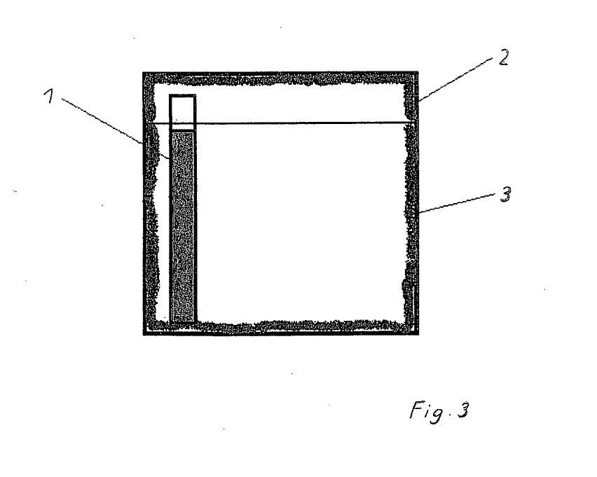

FIG. 3 shows cigarettes in a pack containing flavouring ingredients which are bound in a carrier material which, in turn, is bound in and/or onto the inner side of the cigarette packet;

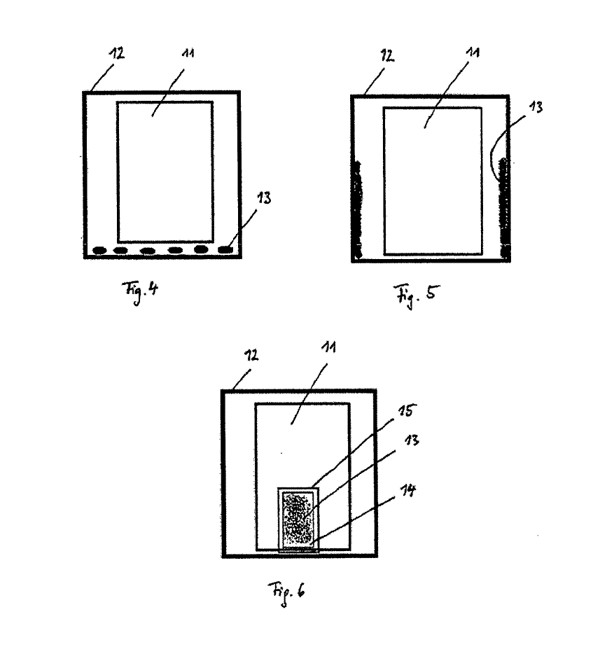

FIG. 4 shows a cellulose-containing tissue arranged inside the packing together with active ingredients bound in a bed of solid granular matter;

FIG. 5 shows a cellulose-containing tissue arranged in a packing where the support material with the bound-in active ingredients are arranged onto and/or in inner side of the packing; and

FIG. 6 shows a cellulose-containing tissue arranged in a packing together with a carrier material with bound-in flavouring ingredients attached to a support material.

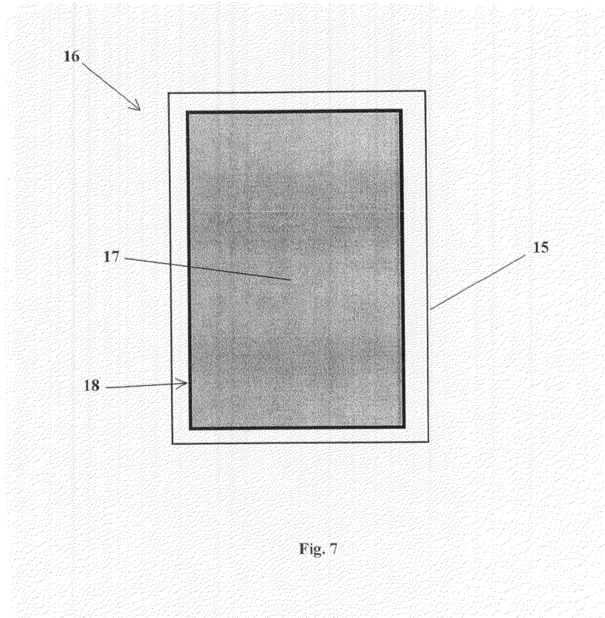

FIG. 7 shows a means for flavouring in accordance with a first embodiment of the invention,

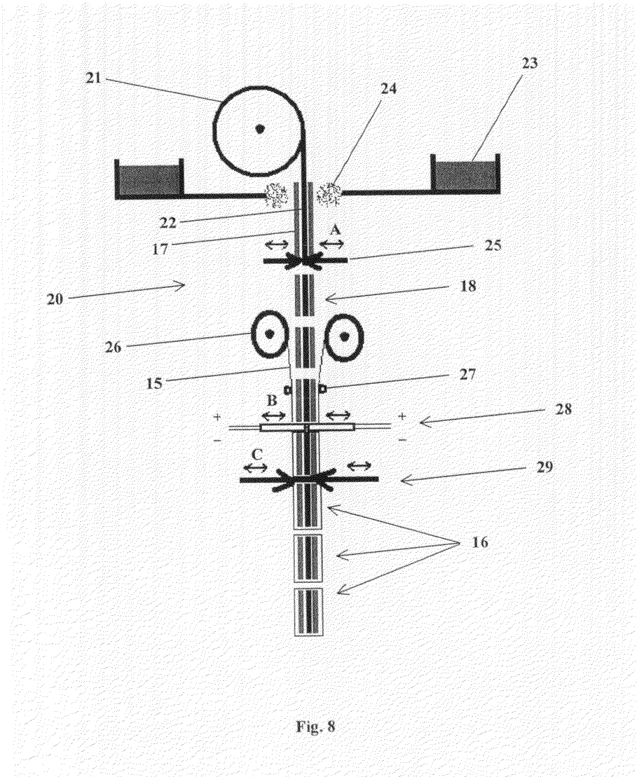

FIG. 8 shows a device for manufacturing the means in FIG. 7,

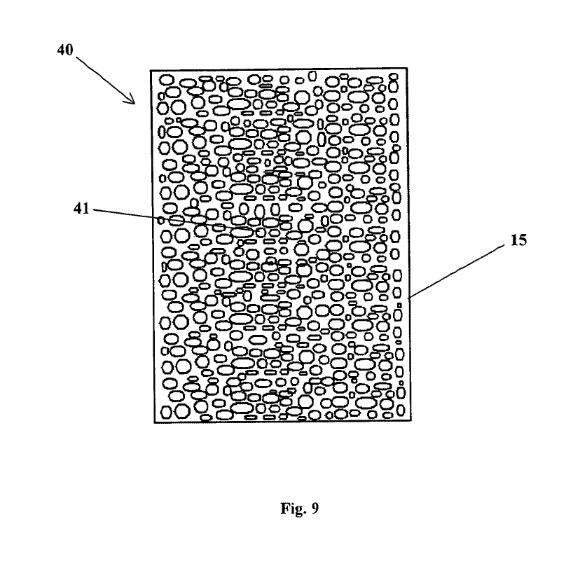

FIG. 9 shows a means for flavouring in accordance with a second embodiment of the invention,

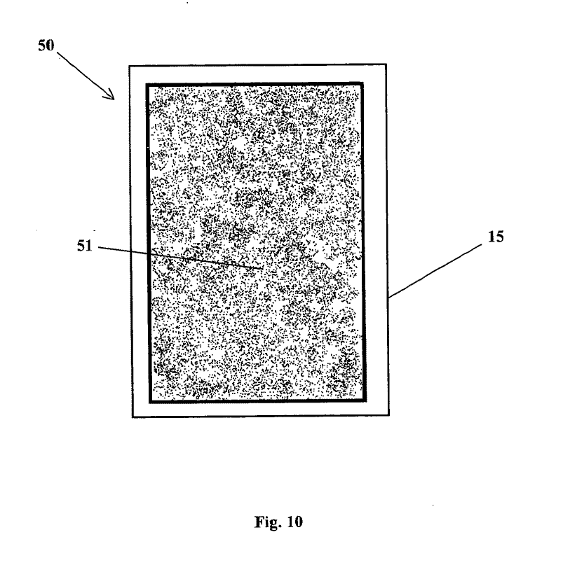

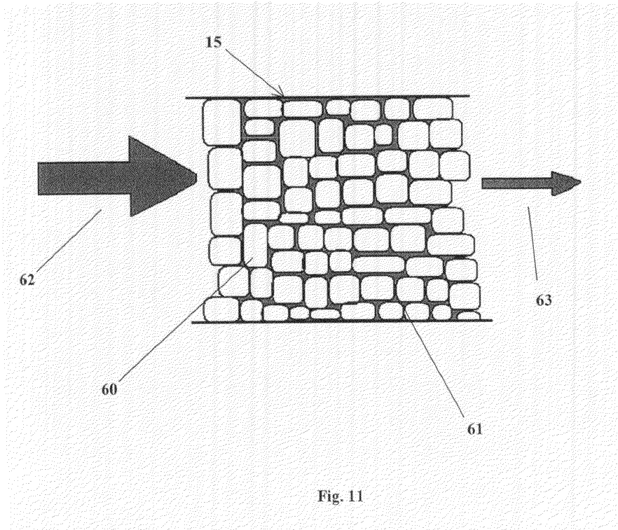

FIG. 10 shows a means for flavouring in accordance with a third embodiment of the invention,

FIG. 11 shows the principle of passage of polar substances through a non-polar enclosure and

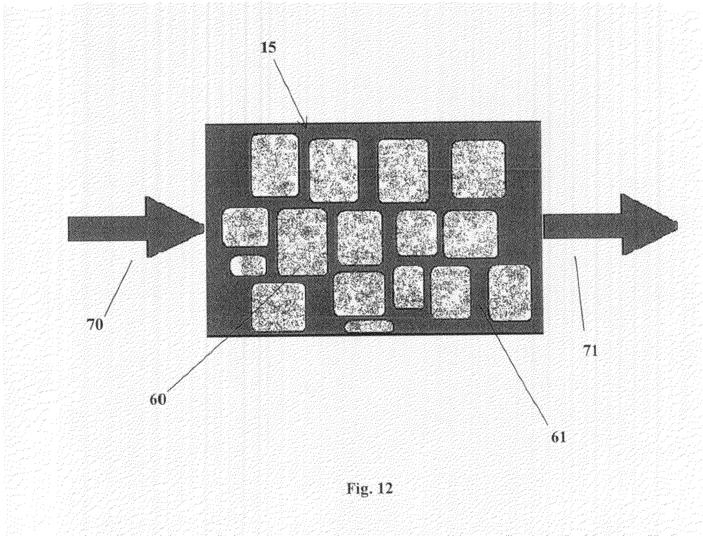

FIG. 12 shows the principle of passage of polar substances that have been absorbed in a non-polar solvent, through a non-polar enclosure.

In a first embodiment, shown schematically in FIG. 1, the invention is described using the example of flavouring cigarettes in a pack. Both the cigarette paper, the filter and above all the tobacco constitute the cellulose-containing product 1 to be flavoured. In the embodiment shown in FIG. 1, flavouring is carried out by introducing the flavour and/or fragrance 3, contained in a solid carrier material, into the package 2 in the form of loose particles. The at least one flavour or fragrance 3 bound in a solid carrier material is released slowly and evenly in the package 2 and penetrates the cellulose-containing product 1, i.e. the cigarette paper, the cigarette filter and the tobacco itself, thus flavouring the cellulose-containing product 1. The at least 1 flavour or fragrance bound in a solid carrier spreads within the pack immediately after the latter is closed and flavours the cigarettes 1 evenly over a long period of time.

Due to the method of the invention it is not necessary to directly flavour the cellulose-containing materials, i.e. the cigarettes including the cigarette paper and the filter in this embodiment, during the manufacturing process. Thus the production process remains completely unaffected and no additional, cost-intensive process steps are required during manufacture.

The high percentage (up to 60 percent by weight) of the flavour and/or fragrance bound in the carrier material allows for intensive flavouring. On the other hand, the at least one flavour and/or fragrance bound in the solid carrier material is released evenly over a long period of time. Thus the cellulose-containing products 1 flavoured in this way can be stored for weeks or even months without any noticeable decrease in the intensity of flavouring.

Another form of flavouring of cellulose-containing products 1 in accordance with the invention is shown schematically in FIG. 2. In the embodiment shown in FIG. 2, the cellulose-containing products 1 are again filter cigarettes packed in hard boxes with gas-tight cellophane wrapping. Here the at least one flavour and/or fragrance 3 bound in a solid carrier material is inserted in the package 2 for the cellulose-containing products 1 by placing the solid carrier material with the at least one flavour or fragrance 3 bound therein on support material 4 or in a support material 4 and putting the support material 4 prepared in this way into the pack 2. A flat material such as paper, cardboard, plastic sheeting or metal foil is especially suitable for use as support material 4. The solid carrier material with the at least one flavour or fragrance 3 bound therein may be sprayed or poured onto the support material 4 in a liquid state. It is also possible to coat support material 4 by immersing support material 4 into a melt of the carrier material and the at least one flavour and/or fragrance bound therein, where the carrier material with the flavour and/or fragrance 3 bound therein, after removal of support material 4 from the melt, will solidify at room temperature or by additional cooling and form a coating.

Porous support materials 4, such as absorptive paper, tissue, non-woven material etc. may also be soaked by inserting them in a melt of the carrier material with at least one flavour or fragrance 3 bound therein. After removal from the melt, the carrier material with the enclosed flavour and/or fragrance 3, penetrated into the support material 4 or adhering to the surface thereof, will solidify as a result of cooling down, thereby being firmly bonded with the support material 4. Further support materials may be ceramics or glass as well as tissue-like products made of textile material, glass, ceramics, metal or plastics fibers.

FIG. 3 shows another schematic embodiment of the process of the invention, for the flavouring of cellulose-containing products 1, where the support material 4 for the insertion or application of the carrier material with at least one flavour or fragrance 3 bound therein constitutes the inner side of the packaging material 2. To coat the inner side of packaging material 2, the most suitable method consists in spraying with a melt of the carrier material with at least one flavour and/or fragrance 3 bound therein and subsequent solidification during cooling-down.

FIGS. 4 through 6 show further embodiments of the method of the invention in a schematical way. In this case, the product is a cellulose-containing tissue 11, a cloth for example, which is inserted into a packing 12. Instead of a flavouring ingredient an active substance 13 such as a therapeutical ingredient is used in that case. In FIG. 4 the active substances 13 are bound in a solid granular carrier material similarly to FIG. 1. On the other hand, FIG. 5 resembles FIG. 3 in that the active substance 13 is bound to a carrier material which is arranged on the inside of a product packing.

FIG. 6 resembles FIG. 2 in that the active substance 13 is embedded in a carrier material arranged in and/or onto a support material 14. To protect delicate products from the carrier material and/or the support material this latter can be provided with a packing 15 of its own which allows the active substance 13 to escape but not the carrier or support material 14. In that case, polyethylene or silicone foil can be used.

It goes without saying that the a.m. packaging material can also be used to protect delicate products 1, 11 from—as shown in FIGS. 1 and 4—granular carrier materials. Or the inner sides of the product packing 2, 12 coated with the carrier material as shown in FIGS. 3 and 6 can be provided with an additional layer of the a.m. packaging material. The important point is that this kind of packaging material can be penetrated by the flavouring ingredients 3 or the active substances 13.

The support material 4, 14 to which the carrier material with the bound-in flavouring (3) or active substances (13) is attached can be arranged within the packing 2, 12 outside or inside the product 1, 11.

To give another example let us mention the application of a packaged fragrance carrier in a PE bag for scenting towels warmed up in a microwave say in an aircraft. So in a test cotton towels (not shown here) contained in a PE bag together with a fragrance embedded in a carrier material and arranged in a support material were warmed for a short time. The towels used were ordinary guest towels, a card-like support material inserted between them in a PE bag, the support material being provided with a thin film of carrier material and fragrance. The microwave was then operated at 800 W for 30 sec. After removing the cloths from the microwave they emitted a much stronger aroma impression than in a comparison test carried out for 30 sec. where no microwave radiation was applied. This shows that the application of energy can significantly accelerate the flavouring process. In the same way, the transmission of an active substance can be accelerated.

The cloths used in the test were dry. When used in a wet state, an additional acceleration can be expected due to a stronger absorption of the microwave radiation.

An essential conclusion is therefore that the energy applied by radiation can be absorbed either by the goods themselves or by the support material or the carrier material or the fragrance itself in order to enhance the release of the fragrance.

Flavouring and the transmission of the active substance can be done in different ways. The carrier materials can be packaged separately in order to protect the products. The packaging can be omitted if such products are insensitive to the carrier materials. The products can be arranged in a packaging. Such packaging of the products can, however, be omitted. In a microwave for example it is not necessary since the microwave housing takes over the function of packaging. Carrier materials can therefore be arranged onto, in or in proximity of the products.

External sealing (not shown here) of the product packaging 2, 12 is of advantage since a decrease of the substance concentration inside the packaging 2, 12 is avoided on the one hand, and the undesirable affection of further adjoining products (not shown here) is excluded on the other. The sealing materials of choice are cellophane or metal foils, they can be arranged as one piece on the product packaging 2, 12.

The method of the invention for a flavouring process by which cellulose-containing products 1 after being packaged can be flavoured indirectly within the packaging material 2 has the following substantial advantages as compared to the well-known direct methods of flavouring of cellulose-containing products during the manufacturing process:
a) The manufacturing process for the products 1 remains unaffected by the flavouring process
b) Flavouring does not start until the assignment of the flavouring ingredients to the cellulose-containing products 1 or the packaging of the cellulose-containing products 1 and thus at a later time than its actual production which means at a stage closer to consumption.
c) By bonding at least one flavour and/or fragrance 3 to a carrier material solid at normal temperature, the flavour/fragrance is released and transferred to the products 1 within the pack over a longer period of time, where the high percentage of flavour and/or fragrance 3 of up to 60% by weight ensures intensive flavouring.
d) The products 1 to be flavoured can be provided with different flavours/fragrances depending on the season, i.e. with refreshing summer scents or stimulating and homey Christmas scents. Such seasonal changes are possible without much preparation work; it is only necessary to change the at least one flavour and/or fragrance 3 introduced into the packages and bound in a carrier material solid at normal temperature.

Similar advantages (therefore not explained here in detail) are achieved with the method of the invention also with respect to active substances 13.

FIG. 7 shows a means of the invention for flavouring in a purely schematic way. More specifically, FIG. 7 shows a means 16 consisting of a support material provided with a carrier material 17 where a fragrance has been absorbed and forms a film shape 18. The film shape 18 is sealed with an enclosure 15 towards the environment so that neither carrier material 17 nor the support material may penetrate to the outside, and the environment cannot come into contact with these materials. The enclosure 15 consists of PE and is impermeable to the fragrance.

The fixture 20 used for the manufacture of this means of the invention is shown purely schematically in FIG. 8. Paper 22 is rolled off a paper roll 21 and provided with a coating of carrier material 17 by spraying a melt 23 of the carrier material 17 and the fragrance dissolved therein as spray 24 onto paper 22. Granulates may be molten to obtain this melt. Alternatively, the melt 23 may also be poured onto the material. After the cooling of the sprayed-on melt, the coated endless paper 22 is cut into pieces as required with the help of the cutting device 25, where cutting knives are guided in the cutting direction A arranged perpendicular to the paper 22, which results in the forming of film shapes.

Subsequently, the film shapes 18 are enclosed by attaching a PE sheeting 15, arranged on supply rolls 26 on both sides of the piece guidance, via pressing rolls 27 to the film shapes 18, and each individual film shape 18 is sealed with the PE sheeting 15 using a heating facility 28. During sealing, the two sheeting sides 15 are thermally connected with each other by pressing together heated punches in the heating facility 28 in a direction perpendicular to the piece guidance. Thus each individual film shape 18 is sealed along its external contour. The webs of PE sheeting formed in this way between the individual film shapes 18 are subsequently separated by another cutting device 29, where cutting knives are again guided in a cutting direction perpendicular to the piece guidance, so that the means 16 of the invention are formed consisting of an enclosure 15 in which the paper 22 provided with coating 17, i.e. a film shape 18, has been absorbed and sealed.

FIGS. 9 and 10 show alternative means 40 and 50 differing from means 16 in that respect that in the case of means 40, a granulate such as Chips 41, and in the case of means 50 a powder, that is a powder 51, is sealed in the enclosure.

This means that with the help of means 16, 40, 50 of the invention, i.e. a combination of film shape 18, granulate 41 or powder 51 with a certain enclosure 15, refill packages can be provided that can be specifically adapted to different applications. Main applications are the delivery of fragrances, flavours and active substances into certain spaces and the delivery of fragrances, flavours and active substances into other materials, such as cellulose-containing substances.

For room temperature applications, an enclosure 15 made of LDPE is preferred, while an enclosure 15 made of HDPE is preferred for temperatures up to 80° C., and an enclosure 15 made of silicone for temperatures in a range from 80° C. to 200° C. Sheeting 15 of approximately 0.5 mm thickness is preferred in this case since it has sufficient permeability combined with acceptable strength. For special applications, the released amount of fragrances, flavours and active substances can be systematically adjusted via the enclosure thickness or the selection of the form or shape of fragrances, flavours and active substances absorbed in a carrier material and/or the use of specific solvents. The release rates may be further increased by introducing energy.

To demonstrate the effect of a solvent, FIGS. 11 and 12 show the differences during a passage of polar substances through PE sheeting 15. FIG. 11 shows that the PE sheeting 15 consists of individual polyolefin particles 60 with interstices 61 between them. Such PE sheeting is not polar. A polar substance 62 located on one side of the PE sheeting 15 such as a fragrance is severely hampered due to its polarity when passing sheeting 15, and only the interstices 61 allow passage of a small amount of substance 63.

FIG. 12 is a purely schematic display of the effect if the intention is to mix the polar substance with a non-polar solvent into a substance 70. The PE sheeting 15 with the polyolefin particles 60 absorbs the substance 70 since the non-polar solvent is dissolved in the polyolefin particles 60. This results in swelling of the polyolefin particles 60, the interstices 61 become larger and the sheeting 15 swells, which is an effect well known from literature. Now the substance 70 can pass through the sheeting basically without any obstructions, and the amount passing through is much bigger than in the case of FIG. 11.

It has become evident what considerable benefits are connected with the present invention. Flavouring and/or transfer of active substances is possible not only in relation to any products, in particular cellulose-containing products, but also to or in any spaces. These spaces may be larger or smaller and do not necessarily have to be isolated towards the flavouring or active substances. In particular, these can be washing machines, dish washers, laundry driers, refrigerators etc.

1. A process for the flavouring of products, in particular cellulose-containing products (1; 11), and/or spaces and/or for the transfer of active substances to products, in particular cellulose-containing products (1; 11), and/or spaces,
wherein
at least one flavour and/or fragrance (3) and/or an active substance (13) bound in a solid carrier material are arranged in and/or at the product (1; 11) and/or in the immediate vicinity of the product (1; 11) and/or within the space.

2. Process in accordance with claim 1,
wherein
the product (1; 11) is contained in a packaging (2; 12) which also contains the solid carrier material.

3. Process in accordance with claim 1, wherein
the solid carrier material with at least one flavour, fragrance (3) or active substance (13) bound therein is used in a particulate form.

4. Process in accordance with claim 3,
wherein
the solid carrier material with at least one flavour, fragrance (3) or active substance (13) bound therein is used in the form of powder, granules, chips or pearls.

5. Process in accordance with claim 1, wherein
the solid carrier material with at least one flavour, fragrance (3) or active substance (13) bound therein is applied onto a support material (4; 14) or is inserted into the support material (4; 14).

6. Process in accordance with claim 5,
wherein
the support material (4; 14) is a flat material or a part of any shape created by basic forming or transforming or combined from several elements, preferably paper, cardboard, plastic sheeting or metal foil, ceramics, glass, non-woven material or tissue or a tissue-like product of textile material, glass, ceramics, metal or plastics fibres.

7. A process in accordance with claim 5, wherein
the solid carrier material with at least one flavour, fragrance (3) or active substance (13) bound therein is applied to the support material (4; 14) or is inserted into the support material (4; 14) from a melt of the carrier material and at least one flavour, fragrance (3) or active substance (13) bound therein, and then solidified by cooling.

8. Process in accordance with claim 7,
wherein
the support material (4; 14) is coated from a melt of the carrier material with at least one flavour, fragrance (3) or active substance (13), where the support material (4; 14) is in particular sprayed, poured or soaked with the melt.

9. Process in accordance with
claim 1, wherein
the inner side of the packaging material (2; 12) itself is used as support material (4; 14) for the application or insertion of the carrier material with the at least one flavour, fragrance (3) or active substance (13).

10. Process in accordance with claim 1, wherein
the active substance (13) used contains at least one substance from the groups of insecticides, antibacterial substances, antifungal or antiputrefactive substances, preservatives, therapeutical substances and substances guiding or controlling animals.

11. Process in accordance with claim 1, wherein
the carrier material is sealed off in relation to product (11) or the environment by means of a enclosure (15) which is impermeable to the carrier material and the support material, but is permeable to the flavouring or active substances (13) in at least one area of the enclosure (15).

12. Process in accordance with claim 11,
wherein
the enclosure (15) in this area consists of paper, polyethylene, in particular modified polyethylene, low density polyethylene or high density polyethylene or silicone sheet.

13. Process in accordance with claim 1, wherein
the packaging (2; 12) comprises a material impermeable to the flavour, fragrance (3) or active substance (13), such as cellophane which is in particular connected as one piece with the packaging (2; 12).

14. Process in accordance with claim 1, wherein
the flavouring of the product or the transfer of active substances (13) to the product (1; 11) or the space is accelerated by exposing the carrier material and/or the product (1, 11) and/or the packaging (2; 12) and/or the space to an energy source, especially to electromagnetic radiation or ultrasound.

15. Process in accordance with claim 14,
wherein
the electromagnetic radiation applied is microwave and/or thermal radiation.

16. Process in accordance with claim 1, wherein
the spaces can be rooms, washing machines, dish washers, laundry driers, refrigerators etc.

17. Means (16; 40; 50) for the flavouring of spaces or products, in particular cellulose-containing products, and/or for transfer of active substances to spaces or products, in particular cellulose-containing products (1; 11), in particular based on the process described in claim 1, wherein
at least one flavour and/or fragrance (3) and/or active substance (13), where the carrier material (17) is enclosed in an enclosure (15) that prevents passage of the carrier material (17) and is permeable to the fragrance, flavour or active substance (13) in at least one section of the enclosure.

18. Means (16) in accordance with claim 17,
wherein
the enclosure may comprise paper, polyethylene (15), in particular modified polyethylene, low density polyethylene or high density polyethylene, or silicone sheet.

19. Means (40, 50) in accordance with claim 17, wherein
the solid carrier material with at least one flavour, fragrance (3) or active substance (13) bound therein is present in particulate form (41, 51), in particular in the form of powder (51) with an external diameter in the range of 10 μm to 100 μm, preferably 25 μm to 50 μm, or in the form of granulate (41), chips or pearls with an outer diameter of up to 10 mm, preferably up to 5 mm.

20. Means (16) in accordance with claim 1, wherein
the solid carrier material (17) with at least one flavour, fragrance (3) or active substance (13) bound therein is applied to or inserted into the support material, where the support material is preferably a flat material or a part of any shape created by basic forming or transforming or combined from several elements, in particular paper (22), cardboard, plastic sheeting or metal foil, ceramics, glass, non-woven material or tissue as well as a tissue-like product from textile material, glass, ceramics, metal or plastics fibres, where the enclosure is in particular impermeable to the support material.

21. Means in accordance with claim 20,
wherein
the support material for the application or insertion of the carrier material with at least one flavour, fragrance or active substance bound therein, constitutes the inner side of the material of the product packaging.

22. Means (16; 40; 50) in accordance with claim 17, wherein
the enclosure (15) has a thickness of 0.1 mm to 2 mm, preferably 0.2 mm to 0.5 mm.

23. Means in accordance with claim 17, wherein
the at least one flavour, fragrance (3) or active substance (13) is dissolved in a non-polar solvent, preferably an isoparaffinic solvent, which preferably has a flash point of greater than 50° C., in particular greater than 60° C., where the volume of solvent related to the entire amount of solvent plus flavour, fragrance (3) or active substance amounts to 0.5% to 50%, in particular 10%.

24. Means in accordance with claim 17, wherein
as active substance (13) at least one substance is used from the group of insecticides, antibacterial substances, antifungal or antiputrefactive substances, preservatives, therapeutic substances and substances guiding or controlling animals.

25. System from at least one means (16) for the flavouring of products and/or transfer of active substances to products in accordance with claim 17 and at least one product, in particular a cellulose-containing product (11).

26. System in accordance with claim 25,
wherein
the system furthermore comprises a packaging (12) that encloses the means (16) and the product (11), where the packaging (12) in particular comprises a material impermeable to the fragrance, flavour (3) or active substance, such as cellophane, which is preferably connected as one piece with the packaging (12).
